(19) United States
(12) Patent Application Publication
(10) Pub. No.: US 2025/0041226 A1
(43) Pub. Date: Feb. 6, 2025

(54) MULTIPARTICULATE ALBACONAZOLE COMPOSITION

(71) Applicant: PALAU PHARMA, S.L.U., Barcelona (ES)

(72) Inventors: MONTSERRAT SURIOL FERRER, Barcelona (ES); MARTA BUSQUETS BAQUÉ, Barcelona (ES)

(21) Appl. No.: 18/722,868

(22) PCT Filed: Dec. 20, 2022
(86) PCT No.: PCT/EP2022/086909

§ 371 Date: Jun. 21, 2024

(30) Foreign Application Priority Data
Dec. 22, 2021 (EP) 21383192.8

(51) Int. Cl. A61K 9/16; A61K 31/517

(52) U.S. Cl. CPC A61K 9/1676; A61K 9/1623; A61K 9/1641; A61K 9/1652; A61K 9/1682; A61K 31/517

(57) ABSTRACT

The present invention provides an oral solid pharmaceutical composition in the form of a multiparticulate composition comprising albaconazole, a process for the preparation of the composition of the invention, and its use in therapy.

### FIELD OF THE INVENTION

The present invention provides an oral solid pharmaceutical composition in the form of albaconazole-containing pellets, a process for the preparation of said composition and its use in therapy.

### BACKGROUND OF THE INVENTION

Albaconazole has been first described in WO 97/05130 A1 as one among a variety of new pyrimidone derivatives with antifungal activity and that it might be used in different therapeutic applications. In said document various oral pharmaceutical compositions containing pyrimidone derivatives, such as tablets, dispersible powders or granules, as well as liquid formulations were described. Also, preparations for vaginal or rectal administration were mentioned. Representative pyrimidone derivatives containing formulations that were mentioned in WO 97/05130 A1 are a tablet formulation, a hard gelatin capsule formulation, a syrup formulation, an aerosol formulation, and two different injection formulations. However, none of these formulations was tested in any way and there is no experimental data in WO 97/05130 A1 for any of the disclosed dosage forms.

In document WO 2008/021049 A2, different crystalline forms of Albaconazole and their use in pharmaceutical formulations were described. However, formulations containing Albaconazole crystalline forms were mentioned only generically without providing any specific example. Hence, there is no information how a specific Albaconazole polymorph might behave in a specific pharmaceutical formulation and again no experimental data has been provided for the disclosed dosage forms.

Document WO 2010/138674 A1 discloses a method for the treatment or prophylaxis of a fungal condition in a patient comprising topically applying to the patient a dosage selected from the group consisting of a nail lacquer, enamel, paint, solution, lotion, cream, gel, aerosol foam and aerosol spray form. The active ingredient is a pyrimidone derivative such as Albaconazole. Pharmaceutical compositions for oral administration are not mentioned in this document.

Pharmaceutical compositions comprising Albaconazole for oral administration have been disclosed and described for example in document Clinical Pharmacology: Advances and Applications 2013:5 23-31. This document describes a comparison between tablet and capsule formulations in order to determine bioavailability, bioequivalence, safety and tolerability. The tested capsule formulation contained microcrystalline cellulose pellets coated with a mixture of Albaconazole in combination with amino methacrylate copolymer, talc, colloidal SiO₂, hydrochloric acid, anhydrous alcohol, and purified water. However, there were no further reports about those formulations and it was not disclosed how to prepare those formulations. Yet, the use of amino methacrylate copolymer hints to the formation of a solid dispersion.

However, there remains the need to provide pharmaceutical compositions for oral administration comprising Albaconazole that are simple in its composition and provide nonetheless an appropriate dissolution behaviour allowing for good to excellent bioavailability. There is also a need to provide such pharmaceutical compositions at large scale for industrial purposes and not be limited to laboratory amounts only.

This is not easily achievable, since Albaconazole is very hydrophobic, has poor flowability and has a tendency to hydrolyze in the presence of aqueous solutions. These physicochemical properties make it very difficult to obtain oral compositions containing albaconazole that have a good solubility profile and good bioavailability.

In order to circumvent the problem of hydrophobicity of albaconazole, the API was first used in its amorphous form when testing different excipients. Simple blends with excipients, such as mannitol, sodium lauryl sulfate, sodium croscarmellose, colloidal anhydrous silica or even mixtures thereof, were tried but resulted in formulations having no wettability or forming agglomerates. Moreover, in dissolution tests, high variability was observed and sometimes even particles remained in solution. In general, those simple blends did not achieve uniform and homogeneous compositions.

Therefore, wet granulation (using water as granulation liquid) was tested to formulate the product. Wet granulation technique was expected to improve the dissolution behaviour and to overcome the problems observed with the simple blends. However, hydrophobicity remained a problem even with the addition of surfactants in the formulations and agglomerations were still observed during dissolution tests.

Since the previous techniques did not provide the desired results, hot melt technology was tried, since it is said that it may improve dissolution in complicated cases. Here, an appropriate excipient was heated to its melting point and then Albaconazole was added to the melt. The formed dispersion was then allowed to cool down in order to obtain a solid dispersion that could be obtained as granules. Excipient Gelucire 44/14 (32-lauroyl macrogolglycerides) was used and showed promising results when used in the proportion Albaconazole:Gelucire 44/14 1:4 with about 87% of Albaconazole dissolved after 45 minutes in 0.1N HCl. These promising results were obtained for a dosage form containing 40 mg Albaconazole. However, higher doses were not feasible because the resulting formulations were too waxy to handle and because the required amount of Gelucire 44/14 was just too high to obtain adequate dosage forms for oral administration.

A solution for obtaining higher doses with hot melt technology was found in using a mixture of Gelucire 44/14 and diluents, such as mannitol, lactose monohydrate or mixtures thereof. However, these compositions, manufactured by hot melt technology, showed problems under defined storage conditions. At 40° C. and 75% relative humidity (RH) after 1 month of storage, the composition changed into a compact waxy something that didn't meet the required specifications and did hardly allow albaconazole to dissolve. Similar issues were observed at 30° C. and 60% RH after 1 month of storage, although to a lesser extent. And even at the mildest condition of 25° C. and 60% RH, a considerable drop of dissolution was observed after 6 months making it nearly impossible to meet the requirements for a dosage form having the desired bioavailability.

### BRIEF DESCRIPTION OF THE INVENTION

The present inventors have now developed a multiparticulate composition comprising pellets coated with an albaconazole-containing composition which is simple in its constitution and easily obtainable at large scale. Said multiparticulate composition provides an Albaconazole formulation with good dissolution behaviour and bioavailability.

In one particular aspect, albaconazole in the albaconazole-containing composition is present in solid form. Preferably, the solid form of albaconazole may be amorphous or crystalline, more preferably crystalline. It has been observed that the multiparticulate composition comprising crystalline Albaconazole under defined stability testing conditions maintains the crystalline solid form of the API, meaning that the crystalline Albaconazole is stable and does not change its crystalline form into another crystalline form.

Also a manufacturing process has been developed to prepare the coated pellets of the invention.

In a first aspect of the present invention refers to a multiparticulate pharmaceutical composition comprising:
a) a plurality of individual, substantially spherical inert particles having a diameter comprised between 300 and 800 μm;
b) a coating on said particles comprising albaconazole, a coating agent and a plasticizer agent.

A second aspect of the present invention provides a process for preparing the multiparticulate pharmaceutical composition according to the first aspect.

The third aspect of the present invention refers to the multiparticulate composition according to the first aspect for use in the treatment of fungal infections.

The fourth aspect of the present invention refers to the use of a composition according to the first aspect for the manufacture of a medicament for the treatment of fungal infections.

The fifth aspect of the present invention refers a method for treating fungal infections by administering to a subject in need thereof of a composition according to the first aspect.

### BRIEF DESCRIPTION OF THE DRAWINGS

FIG. 1 is the XRD diffraction pattern of Albaconazole Form III

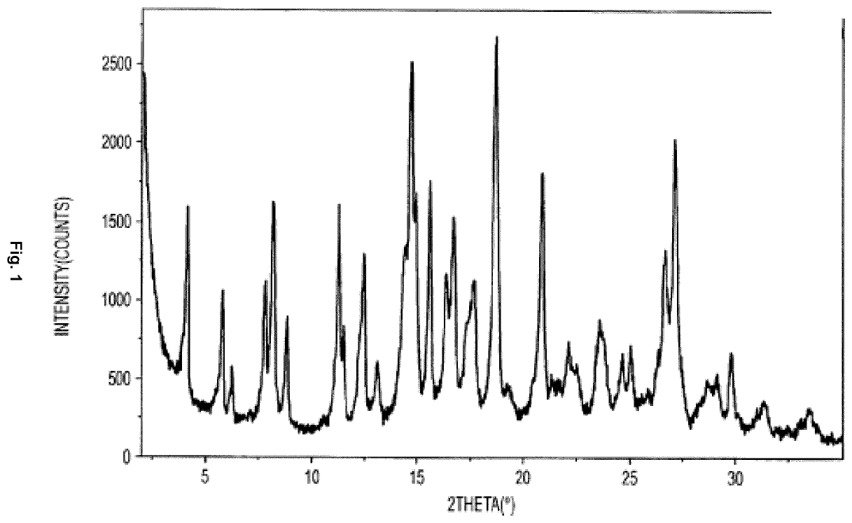

FIG. 2 is the XRD diffraction pattern of Albaconazole Form IV

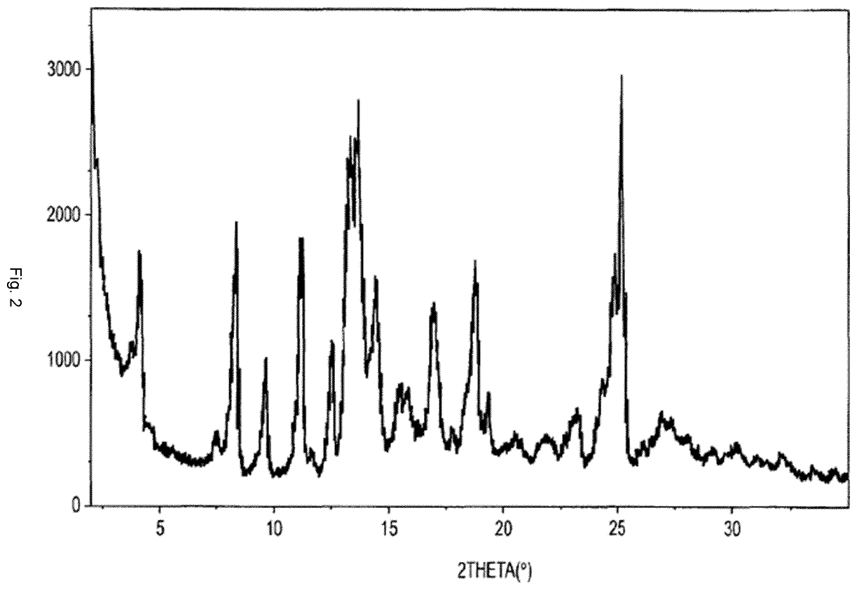

FIG. 3 is the XRD diffraction pattern of Albaconazole Form VI

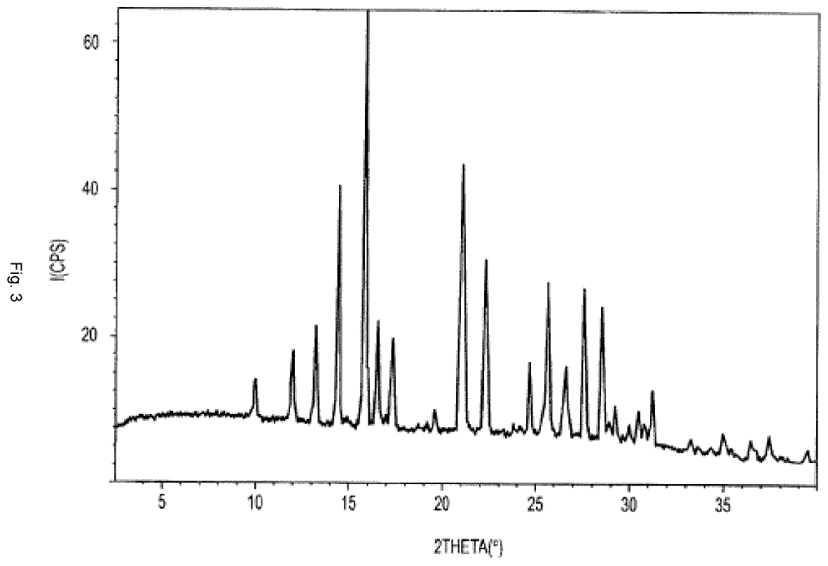

FIG. 4 is the XRD diffraction pattern of Albaconazole amorphous

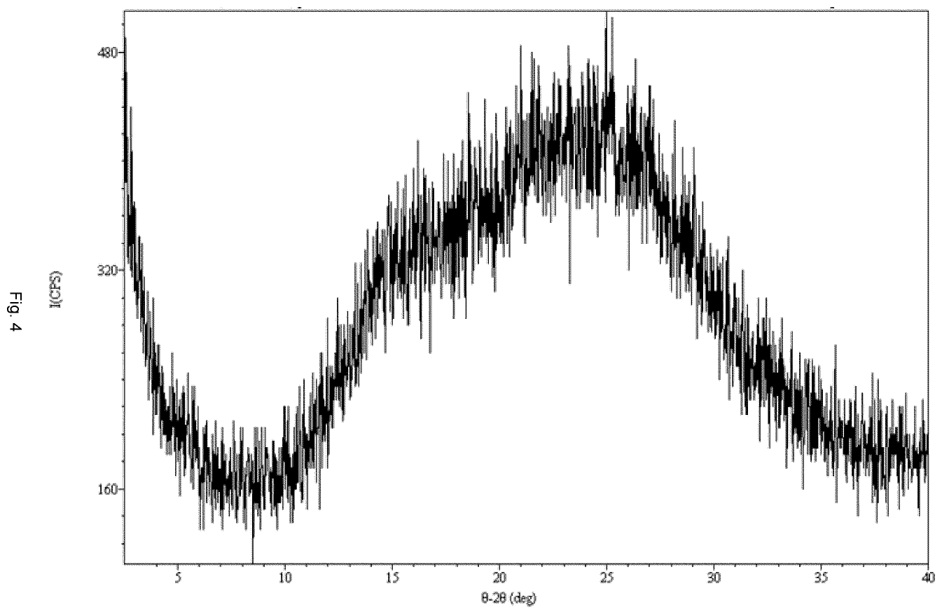

FIG. 5 is a dissolution profile comparing Albaconazole capsules (amorphous) versus Albaconazole capsules (crystalline)

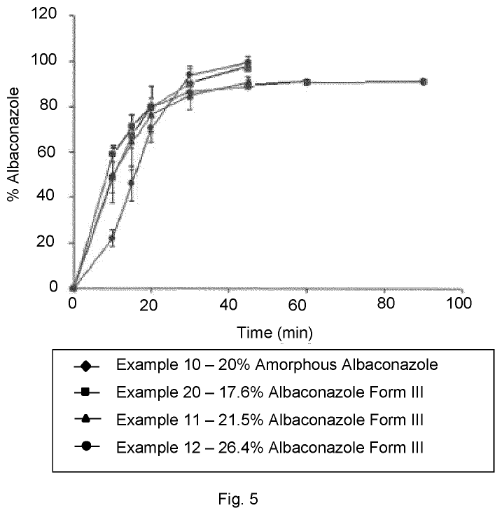

### DEFINITIONS

The term “about” as used herein refers to a statistically meaningful range of a value, typically within 10%. Such a range can lie within experimental error, typical of standard methods used for the measurement and/or determination of a given value or range. In one embodiment, the range is within 5% of the indicated value. In another embodiment, the range is within 1% of the indicated value. In yet another embodiment, the range is within 0.5% of the indicated value.

The term “pharmaceutically acceptable” as used herein refers to those compounds, materials, compositions, and/or dosage forms which are, within the scope of sound medical judgment, suitable for contact with the tissues of animals, in particular human beings, without excessive toxicity, irritation, allergic response, or other problematic complications commensurate with a reasonable benefit/risk ratio.

The term “treating”, as used herein, unless otherwise indicated, includes the amelioration, cure, and/or maintenance of a cure (i.e., the prevention or delay of relapse) of a disease or disorder. Treatment after a disorder has started aims to reduce, alleviate, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse).

The term “multiparticulate pharmaceutical composition” as used herein refers to a pharmaceutical composition in the form of multiple discrete solid units.

The term “coating” as used herein refers to adherence, and/or adsorption, preferable uniformly, of at least one coating material onto a substrate. Preferably the coating material is a thin and uniform film applied onto the substrate.

The term “inert particles”, irrespective of the material used in the different aspects and/or embodiments, as used herein refers to particles that have no therapeutic activity of its own. The particles may be in the form of spheres or pellets.

### DETAILED DESCRIPTION OF THE INVENTION

The inventors of the present invention have surprisingly found that coating substantially spherical inert particles having a diameter comprised between 300 and 800 μm with a mixture comprising albaconazole, a coating agent and a plasticizer agent allows the preparation of an oral solid pharmaceutical composition having good flowability, stability and dissolution.

Thus, the first aspect of the present invention refers to a multiparticulate pharmaceutical composition comprising:
a) a plurality of individual, substantially spherical inert particles having a diameter comprised between 300 and 800 μm;
b) a coating on said particles comprising albaconazole, a coating agent and a plasticizer agent.

In a second aspect, the present invention relates to a process for the preparation of the multiparticulate composition of the first aspect, comprising:
i) providing an aqueous solution of the coating agent and the plasticizer agent,
ii) optionally, adding a surfactant to the solution of step i),
iii) dispersing albaconazole in the solution resulting from step i) of from step ii) if this step is present, to form a suspension,
iv) optionally sieving the suspension of step iii) to remove particles with a particle size above 90 μm,
v) coating a plurality of spherical inert particles having a diameter comprised between 300 and 800 μm with the suspension resulting from step iii), or from step iv) when said step is present,
vi) optionally, drying the product resulting from step v)
vii) optionally, filling capsules with the product resulting from step v).

In an embodiment of the first or second aspects of the present invention, the substantially spherical inert particles, before being coated, have a diameter between 300 and 800 μm, preferably between 400 and 750 μm, more preferably between 450 and 750 μm, more preferably between 500 and 710 μm and most preferably between 580 and 680 μm.

Preferably, the substantially spherical inert particles are neutral inert particles having no acidic or alkaline nature and are selected from the group comprising or consisting of sugar particles, cellulose particles or silicon dioxide particles. More preferably, the substantially spherical inert particles are sugar particles. The advantage of said substantially spherical inert particles is that the specific surface on which Albaconazole is present is increased resulting in improved dissolution of Albaconazole when compared to a simple blend. Sugar particles are preferred because they allow to lower the manufacturing costs of the whole process, so that the final product and compositions comprising the final product can be made available to a broader public.

In the context of the present invention the term “substantially spherical” is used to designate particles having a sphericity factor (Ψ_{w}) comprised between 0.9 and 1.1, more preferably between 0.95 and 1.05, wherein the sphericity factor is defined as the ratio between the surface area of a sphere having the same volume as the particle and the surface area of the particle:

$${Ψ}_{W} = \frac{{d}_{V}^{2}}{{d}_{S}^{2}}$$

where dᵥ and dₛ are the equivalent volume and surface diameter, respectively (Part. Part. Syst. Charact. 1996, 13, 368-373).

In the context of the present invention sugar particles are particles comprising sucrose and starch.

In the context of the present invention cellulose particles are particles comprising microcrystalline cellulose, preferably made of microcrystalline cellulose.

In the context of the present invention silicon dioxide particles are particles comprising silicon dioxide, preferably made of silicon dioxide.

The starch present in the above mentioned sugar particles is selected from the group consisting of natural starches, such as corn starch, maize starch and potato starch and mixtures thereof. Preferably the starch is corn starch.

In another embodiment of the first and/or second aspects of the present invention, the substantially spherical sugar particles comprise sucrose and starch, preferably the sugar particles comprise at least 60% by weight of sucrose and the rest, i.e. up to a maximum of about 40% by weight, being starch, more preferably the sugar particles comprise between 62 and 92% by weight of sucrose and between 8 and 38% by weight of starch, preferably corn starch.

In another embodiment of the first and/or second aspects of the present invention, the coating is prepared from a suspension comprising albaconazole, a coating agent and a plasticizer agent. Preferably, the resulting coated inert particles comprise (i) 3.5 to 30% by weight, preferably 5 to 30% by weight, more preferably 6 to 30% by weight, more preferably 6 to 25% by weight, and most preferably 10 to 25% by weight of albaconazole; (ii) 0.1 to 17% by weight, preferably 2 to 15% by weight, more preferably from 5 to 15% by weight, more preferably from 8 to 14% by weight, and most preferably 9 to 12% by weight of coating agent; and (iii) 0.1 to 5% by weight, preferably 0.5 to 5% by weight, more preferably from 1 to 4% by weight, more preferably from 2 to 3.5% by weight, most preferably from 2.5 to 2.9% by weight of plasticizer agent; the remaining % by weight of the coated inert particle comprising the inert particle as such and optional ingredients. All weight percentages given here are % by weight relating to 100 mg of coated particles.

The coating agent may be any of cellulose ethers, preferably selected from the group comprising or consisting of hydroxypropylmethyl cellulose, methylcellulose, ethylcellulose, hydroxyethyl cellulose, hydroxypropyl cellulose, carboxymethyl cellulose calcium, povidone and other water-soluble povidone-derived polymers or combinations thereof. More preferably, the coating agent is hydroxypropylmethyl cellulose. These coating agents allow to maintain the solid form of Albaconazole.

The plasticizer agent is selected from the group comprising or consisting of polyethylene glycol, polysorbates, triacetin, triethyl citrate or combinations thereof. More preferably, the plasticizer agent is polyethylene glycol. The use of the plasticizer agent helps to obtain a good coating with an improved dissolution profile and liberation of Albaconazole.

In another embodiment of the first and/or second aspects of the present invention, the coated inert particles additionally comprise from 0 to 1% by weight, preferably 0.1 to 1% by weight, more preferably 0.2 to 0.7% by weight, most preferably 0.3 to 0.5% by weight of a surfactant. The surfactant within the above amounts improves the dissolution of Albaconazole. The weight percentages given here are % by weight relating to 100 mg of coated particles.

In another embodiment of the first and/or second aspects of the present invention, the surfactant is sodium lauryl sulfate, polyoxyethylene sorbitan fatty acid esters (known as Tween), sorbitan ethers/esters (also known as Spans) or combinations thereof. Sodium lauryl sulfate (SLS) is preferred because it does not show interaction with the other components of the coating and is inert in this respect.

In another embodiment of the first and/or second aspects of the present invention, albaconazole is used in either amorphous or crystalline form. Preferably, albaconazole in crystalline form is used due to a better stability performance within the coating and a reduced impurity profile over amorphous It has also been observed that crystalline Albaconazole dissolves faster at early times than amorphous Albaconazole. Known crystalline forms of Albaconazole that may be used in the present invention are Forms 1, II, III, IV, V and VI disclosed in EP 2650291 A1, preferably Albaconazole crystalline forms III, IV or VI.

Albaconazole Forms III, IV and VI can be prepared as described in EP 2650291.

Very relevant 2-Theta (±0.2°) peak positions of Form III in a characteristic X-ray powder diffraction (XRPD) pattern (which is described in EP 2650291A1) comprise at least one of 4.08, 5.73, 6.22, 7.77, 8.15, 8.80, 11.25, 11.47, 12.44, 13.09, 15.57, 17.63, 18.66, 20.85, 26.65 and 27.12°. Preferably, crystalline Form III has a characteristic X-ray powder diffraction (XRPD) pattern that may contain at least one 2-theta position selected from the group consisting of those at about 4.08, 5.73, 6.22, 7.77, 8.15, 8.80, 11.25, 11.47, 12.44, 13.09, 14.33, 14.68, 14.89, 15.57, 16.35, 16.68, 17.27, 17.63, 18.66, 19.32, 20.85, 22.12, 22.49, 23.58, 24.63, 25.02, 26.65, 27.12, 28.74, 29.11, 29.81, 31.35, and 33.48+/−0.2.

Very relevant 2-Theta (±0.2°) peak positions of Form IV in a characteristic X-ray powder diffraction (XRPD) pattern (which is described in EP 2650291A1) comprise at least one of 4.15, 7.5, 8.33, 9.61, 11.16, 12.49, 13.29, 13.64, 14.41, 16.90, 18.74, 24.78, and 25.11°. Preferably, crystalline Form IV has a characteristic X-ray powder diffraction (XRPD) pattern that may contain at least one 2-theta position selected from the group consisting of those at about 3.74, 4.15, 7.5, 8.33, 9.61, 11.16, 11.61, 12.49, 13.29, 13.64, 14.41, 15.43, 15.74, 16.90, 17.71, 18.25, 18.74, 19.30, 20.43, 21.78, 23.20, 24.26, 24.78, 25.11, 26.03, 26.86, 27.25, 28.00, 29.05, 30.07, 30.91, and 32.05+/−0.2.

Very relevant 2-Theta (±0.2°) peak positions of Form VI in a characteristic X-ray powder diffraction (XRPD) pattern (which is described in EP 2650291A1) comprise at least one of 10.1, 14.5, 16.0, 21.1, 24.8, and 25.7°. Preferably, crystalline Form VI has a characteristic X-ray powder diffraction (XRPD) pattern that may contain at least one 2-theta position selected from the group consisting of those at about 10.1, 12.1, 13.3, 14.5, 15.0, 16.0, 16.6, 17.0, 17.4, 18.8, 19.2, 19.7, 21.1, 22.3, 23.9, 24.2, 24.8, 25.7, 26.7, 27.6, 28.6, 28.9, 29.3, 29.7, 30.0, 30.5, 30.8, 31.3, 33.3, 33.7, 34.3, 35.0, 35.5, 36.5, 36.7, 37.4, and 39.5+/−0.2.

Most preferably, Albaconazole form Ill is used because it has shown excellent dissolution results combined with low impurities during stability experiments. It has also shown to be the most reliable solid form within the coating on the long term.

As described in EP 2650291, the XRPD pattern for Form III and Form IV was measured at room temperature using a Philips X′Pert diffractometer equipped with a θ/2θ goniometer, a Cu tube working at 50 kV and 40 mA (CuKα radiation, λ=1.5419 A), a divergence slit=¼°, Soller slits=0.04 rad, an anti-scatter slit=¼°, a receiving slit=0.10 mm, and a secondary curved graphite monochromator. Data were collected in the range 2-35° of 2theta using a step-scan technique with a step size=0.02° and a time per step=20 s. Whereas the XRPD pattern for Form VI was measured using either (1) an Inel XRG-3000 diffractometer equipped with a CPS (Curved Position Sensitive) detector with a 20 range of 120°. Real time data were collected using Cu-Kα radiation. The tube voltage and amperage were set to 40 kV and 30 mA, respectively. The monochromator slit was set at 5 mm by 160 μm. The pattern is displayed from 2.5-40°2θ. Samples were prepared for analysis by packing them into thin-walled glass capillaries. Each capillary was mounted onto a goniometer head that is motorized to permit spinning of the capillary during data acquisition. The samples were analyzed for 300 sec. Instrument calibration was performed using a silicon reference standard. Or (2) alternatively, XRPD analyses were performed using a Shimadzu XRD-6000 X-ray powder diffractometer using Cu Kα radiation. The instrument was equipped with a long fine focus X-ray tube. The tube voltage and amperage were set to 40 kV and 40 mA, respectively. The divergence and scattering slits were set at 1° ° and the receiving slit was set at 0.15 mm. Diffracted radiation was detected by a NaI scintillation detector. A θ-2θ continuous scan at 1°/min (0.4 sec/0.02° step) from 2.5 to 40° 2θ was used. The sample was spun at a rate of 25 rpm. A silicon standard was analyzed to check the instrument alignment. Data were collected and analyzed using XRD-6100/7000 v. 5.0. Samples were prepared for analysis by placing them in an aluminum holder with silicon well.

In the context of the present invention, the most preferred coating agent hydroxypropylmethyl cellulose has a viscosity comprised between about 0.5 mPa·s to about 50 mPa·s. Preferably, the hydroxypropylmethyl cellulose has a viscosity comprised between about 1 mPa·s to about 10 mPa·s, more preferably comprised between about 2 mPa·s to about 8 mPa·s, most preferably comprised between about 4 mPa·s to about 6 mPa·s. The viscosity values shown correspond to the measured viscosity of a 2% w/w aqueous solution of hydroxypropylmethyl cellulose at 20° C., measured according to USP method. The preferred hydroxypropylmethyl cellulose may be selected from the group consisting of cellulose ethers graded as E5LV, E15LV, E50LV, and K100LV, preferably K100 LV.

In the context of the present invention, the most preferred plasticizer agent polyethylene glycol has a viscosity at 20° C. of 50% solution of 2700 to 3500 mPas measured according to ISO 6388 and/or a molecular mass calculated of OH value from 16000 to 25000 g/mol. Such a polyethylene glycol is also known as Polyethylene glycol 20000.

In an embodiment of the first or second aspects of the present invention, the multiparticulate pharmaceutical composition can be used to fill capsules or sachets, preferably capsules, or even used for making tablets. Preferably, the composition is filled into hard capsules, such as hard gelatin or HPMC capsules. The size of the capsules depends on the dose to be used, but it may be selected preferably from size No. 1, 00 or 0L.

The dose of Albaconazole to be used may be any dose being therapeutically effective. Preferably, the dose may be from 1 to 400 mg, more preferably from 10 to 200 mg, and even more preferably from 10 to 150 mg. Especially, the dose of Albaconazole may be selected from an amount of 10, 15, 20, 25, 30, 35, 40, 45, 50, 55, 60, 65, 70, 75, 80, 85, 90, 95, 100, 105, 110, 115, 120, 125, 130, 135, 140, 145 or 150 mg and any combination thereof. Most preferred are doses of Albaconazole selected from 20, 25, 40, 50, 75, 80 and/or 100 mg.

The multiparticulate composition of the present invention may comprise further pharmaceutically acceptable excipients within the coating. Suitable excipients include, but are not limited to, binders, diluents, disintegrants, lubricants, sweetening agents, colouring agents and flavouring agents.

Suitable diluents may be selected from the group consisting of cellulose derivatives, such as cellulose powder, microcrystalline cellulose, or silicified microcrystalline cellulose, natural starches, such as maize starch and potato starch, pregelatinized starch, and mixtures thereof.

Suitable binders may be selected from the group consisting of povidone, copovidone, gelatin, polyethylene oxide, alginic acid, modified corn starch, and/or mixtures thereof.

Suitable glidants may be selected from the group consisting of calcium silicate, magnesium silicate, corn starch, colloidal silicon dioxide, silicon hydrogel, talc, sodium stearyl fumarate, and/or mixtures thereof.

Suitable lubricants may be selected from the group consisting of magnesium stearate, calcium stearate, zinc stearate, glyceryl behenate, mineral oil, stearic acid, and/or mixtures thereof.

In an embodiment of the second aspect, the aqueous solution to be used in step iii) comprises a) 2 to 15% by weight, preferably 3 to 10% by weight, and more preferably 4.5 to 7% by weight of coating agent, b) 0.1 to 5% by weight, preferably 0.5 to 3% by weight, and more preferably 1.0 to 2.0% by weight of plasticizer agent and c) 0 to 1% by weight, preferably from 0.1 to 0.8% by weight, and more preferably 0.15 to 0.5% by weight of a surfactant. All percentages here are based on the total weight of the solution obtained in step i) or step ii).

In an embodiment of the second aspect, albaconazole is dispersed in the solution of step i) or step ii), when surfactant (or wetting agent) is present as weII, in a weight ratio of albaconazole to solution, expressed as “weight ratio=kg Albaconazole/kg solution”, comprised between 0.05 and 0.2 preferably between 0.08 and 0.12.

In an embodiment of the second aspect, the coating suspension to be employed in step v) is used at a weight ratio of coating suspension to inert particles, expressed as “ratio=kg coating suspension/kg inert particles”, comprised between 1 and 9, preferably between 2 and 6, most preferably between 2.5 and 4.5. If the weight ratio is below 1, then the resulting coated particles may not be sufficiently homogenous and thus provide a high variability. On the other hand, if the weight ratio is more than 9, there may be problems with the dissolution of Albaconazole, due to the coating layer being too thick.

A further advantage of the composition according to the invention is that no organic solvent is required for its manufacture. This renders the manufacturing process more sustainable and environmentally friendly as well as more economic, since no costly work-up and recycling of organic solvent is required.

An additional advantage of the composition according to the invention is that only one layer of coating with the active ingredient is present and no further layers need to be coated, such as protection layers or other functional layers. Hence, the composition may be maintained simple and straightforward.

In an embodiment of the second aspect, drying of the product resulting from the coating step takes place at a temperature of the product being dried comprised between 35 and 65° C., most preferably between 45 and 55° C.

The composition according to the present invention and/or the dosage forms manufactured from said composition, such as tablets or capsules, may be stored in any form of packaging available on the market. Such packaging may comprise blisters, sachets, bottles or vials. The packaging may include systems or additives to protect the pharmaceutical composition from humidity, light or other detrimental influences, they may include tamper or child resistant systems or features, and they may be made from any material that might be deemed necessary to protect from humidity, light, oxidation or any other detrimental environmental influences. For example, this may comprise standard polyethylene packaging as well as the more protective aluminium blister materials currently on the market.

### EXPERIMENTAL

### Examples

### Example 1

A multiparticulate pharmaceutical composition of the invention was prepared using the following ingredients:

| | Ingredient | Kg/batch | |
|---|---|---|---|
| a) | Sugar spheres 600 | 6.000 | Kg |
| b) | Albaconazole Form III | 1.905 | Kg |
| c) | Hydroxypropyl methyl cellulose (5 mPas) | 1.007 | Kg |
| d) | Polyethylene glycol 20,000 | 0.2573 | Kg |
| e) | Sodium lauryl sulfate | 0.0382 | Kg |
| f) | Water* | 16.89 | Kg |

*Water was removed in a final drying step of the coated particles

The multiparticulate composition was prepared as follows:
1.007 Kg of hydroxypropyl methyl cellulose (5 mPas) and 0.2573 Kg of polyethylene glycol 20,000 were slowly added to a mixing vessel under agitation (1,000-2,000 rpm) to obtain a solution. The solution was left to stand to allow for de-aireation. Then, 0.0382 Kg of sodium lauryl sulfate were added under agitation (500-1,000 rpm) until complete dissolution. To the resulting solution, 1.905 Kg of albaconazole (Form III) were added under agitation during approximately 1 hour to obtain a homogeneous suspension. The suspension was filtered with a 90 μm sieve to eliminate undesired large particles. This resulted in 20.02 Kg of filtered suspension.

A fluid bed coating apparatus (SAR Labortecnic S.A.) was prepared with the following specifications: (Wurster gun/0.8 mm nozzle/textile filter 100 μm). 6.000 Kg of sugar spheres 600 were weighted and heated to 60° C. for 2 minutes. The coating process was then initiated at a fan flow of 313-400 m³/h, an incoming air temperature of 65-85° C., a product temperature of 45-55° C. and an outcoming air temperature of 30-85° C. Coating took place in 4 distinct phases with the following specifications:

### Phase 1:

Pump speed (%, ml/min)=14-15, 23-28
Pulverising pressure (%, bar)=45, 1.94
Time (min)=15 min

### Phase 2:

Pump speed (%, ml/min)=20-21, 31-37
Pulverising pressure (%, bar)=55, 2.94
Time (min)=120 min

### Phase 3:

Pump speed (%, ml/min)=25-28, 43-49
Pulverising pressure (%, bar)=65, 2.94
Time (min)=45 min

### Phase 4:

Pump speed (%, ml/min)=31-33, 52-54
Pulverising pressure (%, bar)=65, 2.94
Time (min)=approx. 235 min (until exhaustion of the coating suspension)

The equipment was emptied and its contents were sieved in a 1,000 μm sieve to obtain 8.905 Kg of product.

TABLE 1
Additional example compositions

| | | Coating | Plasticizer | | | |
|---|---|---|---|---|---|---|
| | Albaconazole | agent | agent | Surfactant | | |
| | % relating | % relating | % relating | % relating | | |
| | to 100 mg | to 100 mg | to 100 mg | to 100 mg | mg | |
| | coated | coated | coated | coated | pellets/ | |
| Example | pellets | pellets | pellets | pellets | capsule | |
| 2 | Amorphous | HPMC | PEG6000 | 0% | 785 | mg |
| | 4.5% | 1.2% | 0.3% | | | |
| 3 | Amorphous | HPMC | PEG20000 | 0% | 770 | mg |
| | 9.1% | 2.5% | 0.6% | | | |
| 4 | Amorphous | HPMC | PEG20000 | SLS | 770 | mg |
| | 6.55% | 10.7% | 2.7% | 0.4% | | |
| 5 | Amorphous | HPMC | PEG20000 | SLS | 667 | mg |
| | 6.35% | 10.7% | 2.7% | 0.4% | | |
| 6 | Amorphous | HPMC | PEG20000 | SLS | 628 | mg |
| | 16.12% | 10.7% | 2.7% | 0.4% | | |
| 7 | Amorphous | HPMC | PEG400 | SLS | 800 | mg |
| | 6.43% | 10.7% | 2.7% | 0.4% | | |
| 8 | Amorphous | HPMC | PEG20000 | SLS | 770 | mg |
| | 6.4% | 10.7% | 2.7% | 0.4% | | |
| 9 | Amorphous | HPMC | PEG20000 | SLS | 527 | mg |
| | 18.97% | 10.7% | 2.7% | 0.4% | | |
| 10 | Amorphous | HPMC | PEG20000 | SLS | 500 | mg |
| | 20.0% | 10.7% | 2.7% | 0.4% | | |
| 11 | Form III | HPMC | PEG20000 | SLS | 465 | mg |
| | 21.5% | 11.5% | 2.86% | 0.46% | | |
| 12 | Form III | HPMC | PEG20000 | SLS | 567 | mg |
| | 26.4% | 14% | 3.5% | 0.56% | | |
| 13 | Form III | HPMC | PEG20000 | SLS | 238 | mg |
| | 21.01% | 11.18% | 2.79% | 0.45% | | |
| 14 | Form III | HPMC | PEG20000 | SLS | 47.6 | mg |
| | 21.01% | 11.18% | 2.79% | 0.45% | | |
| 15 | Form III | HPMC | PEG20000 | SLS | 229.5 | mg |
| | 21.79% | 11.59% | 2.90% | 0.46% | | |
| 16 | Form III | HPMC | PEG20000 | SLS | 459 | mg |
| | 21.79% | 11.59% | 2.90% | 0.46% | | |
| 17 | Form III | HPMC | PEG20000 | SLS | 679.3 | mg |
| | 14.72% | 7.83% | 1.96% | 0.31% | | |
| 18 | Form III | HPMC | PEG20000 | SLS | 339.7 | mg |
| | 14.72% | 7.83% | 1.96% | 0.31% | | |
| 19 | Form III | HPMC | PEG20000 | SLS | 518 | mg |
| | 19.31% | 10.27% | 2.57% | 0.41% | | |
| 20 | Form III | HPMC | PEG20000 | SLS | 489 | mg |
| | 17.6% | 10.7% | 2.7% | 0.4% | | |
| Comparative | Amorphous | HPMC | 0% | SLS | 639 | mg |
| example 1 | 15.65% | 10.7% | | 0.4% | | |

HPMC: hydroxypropylmethyl cellulose
PEG400: polyethylene glycol with an average molecular mass of about 400 g/mol
PEG6000: polyethylene glycol with an average molecular mass of about 6,000 g/mol
PEG20000: polyethylene glycol with an average molecular mass of about 20,000 g/mol
SLS: sodium lauryl sulfate

TABLE 2
Results

| Example | Dose | | Capsule size | Dissolution test |
|---|---|---|---|---|
| 2 | 35 | mg | 00 | n.d. |
| 3 | 70 | mg | 00 | 54% |
| 4 | 50 | mg | 00 | 80% |
| 5 | 42 | mg | 00 | 88% |
| 6 | 101 | mg | 00 | 96.2% |
| 7 | 51 | mg | 00 | 64% |
| 8 | 49 | mg | 00 | 83% |
| 9 | 100 | mg | 00 | 90% |
| 10 | 100 | mg | a) 0L | a) 96.7% |
| | | | b) 00 | b) 97.0% |
| 11 | 100 | mg | 0L | 90.6% |
| 12 | 150 | mg | 0L | 90.9% |
| 13 | 50 | mg | 0L | 92% |
| 14 | 10 | mg | 0L | 87.5% |
| 15 | 50 | mg | 0L | 98.1% |
| 16 | 100 | mg | a) 0L | a) 93.1% |
| | | | b) 00 | b) 94.3% |
| 17 | 100 | mg | 00 | 97.3% |
| 18 | 50 | mg | 1 | 98.1% |
| 19 | 100 | mg | 0L | 96% |
| 20 | 100 | mg | 0L | 95% |
| Comparative | 100 | mg | 00 | 50-60% |
| example 1 | | | | |

Dissolution test: HCl 0.1N, 45 minutes; preferably >70% of dissolved Albaconazole

TABLE 3
Stability experiments with selected examples

| | | | | Impurities |
|---|---|---|---|---|
| | | | | (related |
| | | Dissolution at | Impurities | substances) at |
| | | t = 12 months at | (related | t = 12 months |
| | Dissolution at | a) 25° C./60% RH | substances) at | a) 25° C./60% RH |
| Example | t = 0 months | b) 30° C./65% RH | t = 0 months | b) 30° C./65% RH |
| 11 | 96.7% | a) 90.8% | 0.48 | a) 0.625 |
| | | b) 96.3% | | b) 0.656 |
| 12 | 90.6% | a) 90.1% | 0.179 | a) 0.22 |
| | | b) n.d. | | b) 0.218 |
| 14 | 92% | a) 91.7% | 0.178 | a) 0.206 |
| | | b) 88.3% | | b) 0.193 |

RH: relative humidity
n.d.: not determined
Dissolution was tested as indicated above, confer to table 2
The term “Related substances” refers to total related substances determined by HPLC.
Pellets according to examples 11, 12 and 14 were prepared, then filled into the corresponding capsules (see table 1) and finally packaged in aluminum/aluminum single dose containers and subjected to ICH stability conditions of 25° C./60% RH and 30° C./65% RH.

### Comparative Example 2

An Albaconazole composition using wet granulation was prepared. For a capsule composition having a net weight of 300 mg, 40 mg of Albaconazole where mixed with 224 mg of mannitol and 30 mg of sodium croscarmellose and the resulting mixture was granulated with water containing 6 mg of sodium lauryl sulfate. After manually sieving, granules were filled into gelatine capsules size 1. Dissolution test in HCl 0.1N was performed and after 45 minutes agglomerates were still observed demonstrating insufficient dissolution behaviour.

### Comparative Example 3

Gelucire 44/14 (32-lauroyl macrogolglycerides) was heated to 65° C. and once it had melted, Albaconazole was added slowly under stirring. The dispersion of Albaconazole in the melted excipient was left to cool down to room temperature. A final solid granule was obtained and was sieved through a 500 μm sieve and filled into gelatine capsules size 1 to have 40 mg Albaconazole/capsule. Albaconazole:Gelucire 44/14 proportions of 1:2, 1:3 and 1:4 were prepared and their dissolution was determined in HCl 0.1N after 45 minutes. The preparation with a proportion of 1:2 and 1:3 showed still agglomerates after 45 minutes and thus insufficient dissolution behaviour. The preparation with a proportion of 1:4 dissolved correctly and about 87% of Albaconazole dissolved after 45 minutes. However, higher doses of Albaconazole per capsules could not be prepared because the high amount of the excipient rendered the whole composition too waxy.

### Comparative Example 4

A composition of Albaconazole, Gelucire 44/14, Mannitol and lactose monohydrate was prepared in the proportion Albaconazole:Gelucire 44/14:Mannitol:Lactose monohydrate=1:2.5:2.5:2.5, in order to reduce the amount of Gelucire 44/14. To this end, Albaconazole was blended with Mannitol and lactose monohydrate and then Gelucire was added. The mixture was heated to 65° C. under stirring in order to melt Gelucire and to obtain eventually an Albaconazole dispersion. The resulting dispersion was cooled down to room temperature and sieved through a 200 μm sieve. A final solid granule was obtained and filled into gelatine capsules size 00 to obtain 80 mg Albaconazole/capsule. The thus obtained capsules were packaged in a single dose container composed of white paper (60 g/m²), aluminium (20 μm) and polyethylene (30 g/m²) and then subjected to defined stability studies.

Dissolution test in HCl 0.1N after 45 minutes revealed the following:
after 6 months at 25° C./60% RH, the dissolved Albaconazole dropped to about 64%
after 1 month at 30° C./60% RH, the dissolved Albaconazole dropped to about 25% which is not acceptable
after 1 month at 40° C./75% RH, the dissolved Albaconazole dropped to about 6% which is not acceptable, and the content of the capsule turned into a compact waxy material.

### Analytical Methods:

### 1) Dissolution Test:

Analytical procedure: Ph. Eur. <2.9.3>; UVNis

### Test Conditions:

Paddle apparatus.
Stirring speed: 100 rpm.
Dissolution medium: 0.1 N Hydrochloric acid.

0.1 N Hydrochloric acid: using a pipette, transfer 8.5 ml of hydrochloric acid to a 1,000 ml calibrated flask and dilute to volume with water HPLC grade.
Volume of dissolution medium: 900 ml.
Dissolution time: 45 min.
Temperature of dissolution medium: 37±0.5° C.

### 2) Related Substances:

Analytical procedure: Ph. Eur. <2.2.29>; HPLC

### Chromatographic System:

Column: Luna C18 (2) 5 μm, 150×4.6 mm.
Mobile phase: Acetonitrile/0.5% phosphoric acid as following:

| Time (min) | % Acetonitrile | % 0.5% phosphoric acid |
|---|---|---|
| 0 | 30 | 70 |
| 6.0 | 40 | 60 |
| 15.0 | 40 | 60 |
| 22.0 | 90 | 10 |
| 23.0 | 30 | 70 |
| 29.0 | 30 | 70 |

0.5% phosphoric acid: using a pipette, transfer 5 ml of o-phosphoric acid (85%) to a 1,000 ml calibrated flask and dilute to volume with water HPLC grade.
Flow: 1.0 ml/min.
Wavelength detection: 210 nm.
Injection volume: 20 μl.
Column temperature: 30° C.
Retention time of Albaconazole: approx. 20.0 min.
Chromatogram time: 29 min.

Test solution: Grind the content (pellets) of, at least, 5 capsules, from the obtained powder weight, accurately, about 150 mg and transfer to a 25 ml calibrated flask. Add about 20 ml of acetonitrile HPLC grade and apply the ultrasound during approximately 10 min. Allow to cool and dilute to volume with acetonitrile HPLC grade. Filter through a 0.45 μm Millex HV-PVDF filter or similar, discard the first drops of filtrate (perform in triplicate).

Individual related substances that are lower than the quantification limit (Quantification limit: QL=0.015%), and any peak corresponding to placebo have not been quantified. The compositions of the invention may be used in the treatment of fungal infections in a patient by orally administering said compositions to said patient.

1. A multiparticulate pharmaceutical composition comprising:
a) a plurality of individual, substantially spherical inert particles having a diameter comprised between 300 and 800 μm;
b) a coating on said particles comprising albaconazole, a coating agent and a plasticizer agent.

2. The multiparticulate composition according to claim 1, wherein the substantially spherical inert particles, before being coated, have a diameter between 300 and 800 μm, preferably between 400 and 750 μm, more preferably between 450 and 750 μm, more preferably between 500 and 710 μm and most preferably between 580 and 680 μm.

3. The multiparticulate composition according to any one of the preceding claims, wherein the substantially spherical inert particles are selected from the group comprising or consisting of sugar particles, cellulose particles or silicon dioxide particles; preferably wherein the substantially spherical inert particles are sugar particles and comprise sucrose and starch, preferably at least 60% by weight of sucrose and up to 40% by weight of starch, preferably corn starch.

4. The multiparticulate composition according to any one of the preceding claims, wherein the coating comprises, in % relating to 100 mg of coated particles, (i) 3.5 to 30% by weight, preferably 5 to 30% by weight, more preferably 6 to 30% by weight, more preferably 6 to 25% by weight, and most preferably 10 to 25% by weight of albaconazole; (ii) 0.1 to 17% by weight, preferably 2 to 15% by weight, more preferably to 15% by weight, more preferably 8 to 14% by weight, and most preferably 9 to 12% by weight of coating agent; and (iii) 0.1 to 5% by weight, preferably 0.5 to 5% by weight, more preferably 1 to 4% by weight, more preferably 2 to 3.5% by weight, and most preferably 2.5 to 2.9% by weight of plasticizer agent.

5. The multiparticulate composition according to any one of the preceding claims wherein, the coating additionally comprises, in % relating to 100 mg of coated particles, from 0 to 1% by weight, preferably from 0.1 to 1% by weight, more preferably from 0.2 to 0.7% by weight, and most preferably from 0.3 to 0.5% by weight of a surfactant; and wherein the surfactant is sodium lauryl sulfate, polyoxyethylene sorbitan fatty acid esters, sorbitan ethers/esters or combinations thereof, preferably is sodium lauryl sulfate.

6. The multiparticulate composition according to any one of the preceding claims, wherein albaconazole is used in solid form selected from the group comprising or consisting of crystalline Form III, crystalline Form IV, crystalline Form VI or amorphous Form; preferably wherein albaconazole is used in crystalline Form III.

7. The multiparticulate composition according to any one of the preceding claims, wherein the coating agent is selected from the group comprising or consisting of hydroxypropylmethyl cellulose, methylcellulose, ethylcellulose, hydroxyethyl cellulose, hydroxypropyl cellulose, carboxymethyl cellulose calcium, povidone and other water-soluble povidone-derived polymers or combinations thereof.

8. The multiparticulate composition according to any one of the preceding claims, wherein the plasticizer agent is selected from the group comprising or consisting of polyethylene glycol, polysorbates, triacetin, triethyl citrate or combinations thereof.

9. A pharmaceutical composition comprising capsules containing a multiparticulate composition according to any one of claims 1 to 8.

10. A process for the preparation of the multiparticulate composition according to any one of claims 1 to 8 comprising:
i) providing an aqueous solution of a coating agent and a plasticizer agent,
ii) optionally, adding a surfactant to the solution of step i),
iii) dispersing albaconazole in the solution resulting from step i) or from step ii) if this step is present, to form a suspension,
iv) optionally sieving the suspension of step iii) to remove particles with a particle size above 90 μm,
v) coating a plurality of spherical inert particles having a particle size comprised between 300 and 800 μm with the suspension resulting from step iii), or from step iv) when said step is present,
vi) optionally, drying the product resulting from step v),
vii) optionally, filling capsules with the product resulting from step v).

11. The process according to claim 10, wherein the aqueous solution to be used in step iii) comprises a) 2 to 15% by weight, preferably 3 to 10% by weight, and more preferably 4.5 to 7% by weight of coating agent, b) 0.1 to 5% by weight, preferably 0.5 to 3% by weight, and more preferably 1.0 to 2.0% by weight of plasticizer agent and c) 0 to 1% by weight, preferably from 0.1 to 0.8% by weight, and more preferably 0.15 to 0.5% by weight of a surfactant.

12. The process according to any one of claims 10 to 11, wherein albaconazole is dispersed in the solution of step i) or ii) so that the weight ratio of albaconazole to solution, expressed as “weight ratio=kg Albaconazole/kg solution”, is comprised between 0.05 and 0.2 preferably between 0.08 and 0.12.

13. The process according to any one of claims 10 to 12, wherein the coating suspension to be employed in step v) is used at a weight ratio of coating suspension to inert particles, expressed as “ratio=kg coating suspension/kg inert particles”, comprised between 1 and 9, preferably between 2 and 6, most preferably between 2.5 and 4.5.

14. The process according to any one of claims 10 to 13, wherein the step vi) of drying of the product resulting from the coating step v) takes place at a temperature of the product being dried comprised between 35 and 65° C., most preferably between 45 and 55° C.

15. A composition as defined in any one of claims 1 to 8 for use in the treatment of fungal infections.

16. Use of a composition as defined in any one of claims 1 to 8 for the manufacture of a medicament for the treatment of fungal infections.

17. Method for treating fungal infections by administering to a subject in need thereof of a composition as defined in any one of claims 1 to 8.
